(19) United States
(12) Patent Application Publication
(10) Pub. No.: US 2020/0038349 A1
(43) Pub. Date: Feb. 6, 2020

(54) USE OF N-ACETYLCYSTEINE AMIDE IN THE TREATMENT OF PENETRATING HEAD INJURY

(71) Applicant: Sentient Lifesciences, inc., New York, NY (US)

(72) Inventor: Glenn A. Goldstein, New York, NY (US)

(21) Appl. No.: 16/294,819

(22) Filed: Mar. 6, 2019

Related U.S. Application Data
(63) Continuation of application No. 14/328,225, filed on Jul. 10, 2014, now abandoned.
(60) Provisional application No. 61/891,034, filed on Oct. 15, 2013.
(60) Provisional application No. 61/858,871, filed on Jul. 26, 2013.
(60) Provisional application No. 61/844,653, filed on Jul. 10, 2013.

(51) Int. Cl. A61K 31/16

(52) U.S. Cl. CPC A61K 31/16

(57) ABSTRACT

This disclosure describes methods of use for N-acetylcysteine amide for the treatment of penetrating head injury.

### RELATED APPLICATIONS

This application is a continuation of U.S. application Ser. No. 14/328,225, filed Jul. 10, 2014 which claims priority from U.S. Provisional Patent Application No. 61/844,653, filed on Jul. 10, 2013; U.S. Provisional Patent Application No. 61/858,871, filed on Jul. 26, 2013; and U.S. Provisional Patent Application No. 61/891,034, filed on Oct. 15, 2013; each of which is incorporated herein in its entirety.

### BACKGROUND

There is a need in the art for compounds and therapeutic aspects to treat pathogenesis associated with penetrating head injury. There is a specific needs for therapeutics to increase the viability of neuronal cells subject to penetrating head injury. The disclosure below provides such therapeutics.

### SUMMARY

The disclosure provides a method of treating penetrating head injury in a subject in need thereof comprising administering to the subject a composition comprising a therapeutically effective amount of N-acetylcysteine amide (NACA). In one embodiment, the composition further comprises a pharmaceutically acceptable salt or excipient.

In another embodiment, the treatment of the penetrating head injury comprises penetration of the skin, the outer layer of the skull, the inner layer of the skull, the dura mater or the brain. In other embodiments, the penetration is caused by a gunshot.

In another embodiment, the NACA is administered systemically. According to these embodiments, the NACA is administered intraperitoneally, intravenously, orally or transdermally. In another embodiment, the NACA is administered directly onto or into a wound caused by the penetrating head injury.

In certain embodiments, the subject is a mammal. The mammal can be a human.

In other embodiments, the NACA is administered at between 5 and 10,000; between 75 and 600; or between 200 and 400 mg/kg to the subject.

In certain embodiments, the brain tissue comprises synaptic cells. According to these embodiments, RCR and/or respiration rate is increased in the synaptic cells. In other embodiments, the brain tissue comprises non-synaptic cells. According to these embodiments, RCR and/or respiration rate is increased in the non-synaptic cells. In other embodiments, the non-synaptic cells comprise neuronal soma cells or glial cells.

### BRIEF DESCRIPTION OF THE DRAWINGS

FIG. 1 shows four graphs showing the relative occurrence or abundance of substances found using immunohistochemistry.

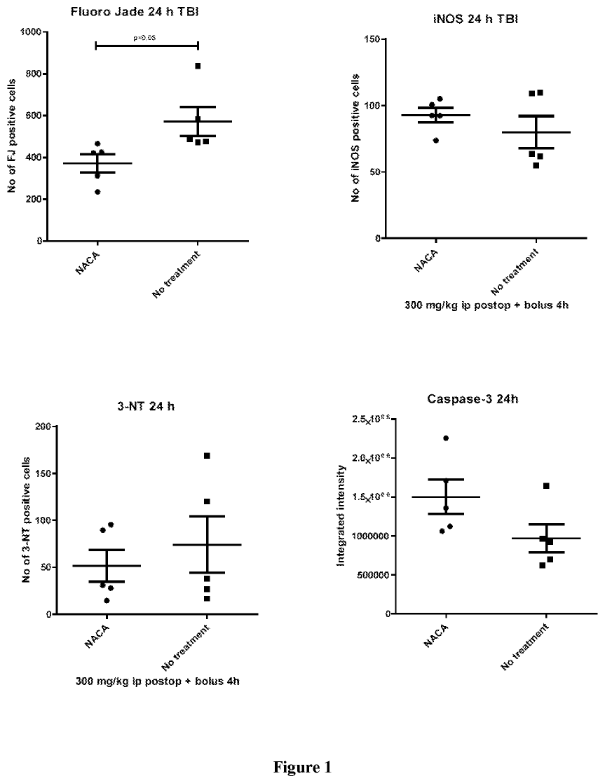

### DETAILED DESCRIPTION

The present invention provides the use of N-acetylcysteine amide (NAC amide or NACA) or derivatives thereof, or a physiologically acceptable derivative, salt, or ester thereof, to treat disorders, conditions, pathologies and diseases that result from, or are associated with penetrating head injury. NACA and its derivatives are provided for use in methods and compositions for improving and treating penetrating head injury.

As used herein, a “subject” within the context of the present invention encompasses, without limitation, mammals, e.g., humans, domestic animals and livestock including cats, dogs, cattle and horses. A “subject in need thereof” is a subject having one or more manifestations of disorders, conditions, pathologies, and diseases as disclosed herein in which administration or introduction of NAC amide or its derivatives would be considered beneficial by those of ordinary skill in the art.

“Therapeutic treatment” or “therapeutic effect” means any improvement in the condition of a subject treated by the methods of the present invention, including obtaining a preventative or prophylactic effect, or any alleviation of the severity of signs or symptoms of a disorder, condition, pathology, or disease or its sequelae, including those caused by other treatment methods (e.g., inflammation), which can be detected by means of physical examination, laboratory, or instrumental methods and considered statistically and/or clinically significant by those skilled in the art.

“Prophylactic treatment” or “prophylactic effect” means prevention of any worsening in the condition of a subject treated by the methods of the present invention, as well as prevention of any exacerbation of the severity of signs and symptoms of a disorder, condition, pathology, or disease or its sequelae, including those caused by other treatment methods (e.g., chemotherapy and radiation therapy), which can be detected by means of physical examination, laboratory, or instrumental methods and considered statistically and/or clinically significant by those skilled in the art.

In another aspect of the present invention, NAC amide is used in the treatment and/or prevention of disorders involving, hair, nails, and mucosal surfaces when applied topically. In accordance with the invention, compositions for topical administration are provided that include (a) NAC amide, or derivatives thereof, or a suitable salt or ester thereof, or a physiologically acceptable composition containing NAC amide or its derivatives; and (b) a topically acceptable vehicle or carrier. The present invention also provides a method for the treatment and/or prevention of cosmetic conditions and/or dermatological disorders that entails topical administration of NAC amide- or NAC-amide derivative-containing compositions to an affected area of a patient.

Another aspect of the present invention provides a compound of the formula I:

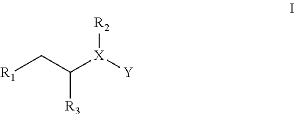

wherein: R₁ is OH, SH, or S—S—Z; X is C or N; Y is NH₂, OH, CH₃—C═O, or NH—CH₃; R.sub.2 is absent, H, or ═O R₃ is absent or

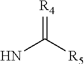

wherein: R₄ is NH or O; R₅ is CF₃, NH₂, or CH₃

and wherein: Z is

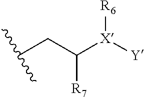

with the proviso that if R₁ is S—S—Z, X and X′ are the same, Y and Y′ are the same, R₂ and R₆ are the same, and R₃ and R₇ are the same.

The present disclosure also provides a NAC amide compound and NAC amide derivatives comprising the compounds disclosed herein. Other derivatives are disclosed in U.S. Pat. No. 8,354,449, incorporated by reference in its entirety.

In another aspect, a process for preparing an L- or D-isomer of the compounds of the present invention are provided, comprising adding a base to L- or D-cystine diamide dihydrochloride to produce a first mixture, and subsequently heating the first mixture under vacuum; adding a methanolic solution to the heated first mixture; acidifying the mixture with alcoholic hydrogen chloride to obtain a first residue; dissolving the first residue in a first solution comprising methanol saturated with ammonia; adding a second solution to the dissolved first residue to produce a second mixture; precipitating and washing the second mixture; filtering and drying the second mixture to obtain a second residue; mixing the second residue with liquid ammonia and an ethanolic solution of ammonium chloride to produce a third mixture; and filtering and drying the third mixture, thereby preparing the L- or D-isomer compound.

In some embodiments, the process further comprises dissolving the L- or D-isomer compound in ether; adding to the dissolved L- or D-isomer compound an ethereal solution of lithium aluminum hydride, ethyl acetate, and water to produce a fourth mixture; and filtering and drying the fourth mixture, thereby preparing the L- or D-isomer compound.

Another aspect of the invention provides a process for preparing an L- or D-isomer of the compounds disclosed herein, comprising mixing S-benzyl-L- or D-cysteine methyl ester hydrochloride or O-benzyl-L- or D-serine methyl ester hydrochloride with a base to produce a first mixture; adding ether to the first mixture; filtering and concentrating the first mixture; repeating steps (c) and (d), to obtain a first residue; adding ethyl acetate and a first solution to the first residue to produce a second mixture; filtering and drying the second mixture to produce a second residue; mixing the second residue with liquid ammonia, sodium metal, and an ethanolic solution of ammonium chloride to produce a third mixture; and filtering and drying the third mixture, thereby preparing the L- or D-isomer compound.

Penetrating Head Wound

According to certain embodiments, NACA and derivatives thereof are used in the treatment of penetrating head wounds. In certain embodiments, the injuries or wounds are caused by trauma or surgery. Penetrating head wounds include wounds that produce any degree of penetration into the head. The penetration can be any one or more layers of the head of a subject. These layers can include the skin, underlying muscle, connective tissue, bone, ear, ear canal, eye, eye socket, mouth, teeth, jaw, skull and brain. Parts of the brain that can be penetrated according to the penetrating head wounds described herein include the cerebellum, medulla, pons, hypothalamus, thalamus, optic tectum, neocortex, basal ganglia, olfactory bulb and cerebral cortex.

In certain embodiments, administration of NACA reduces symptoms associated pathology associated with the penetrating head wound. The symptoms associated pathology can present as symptoms including subdural hematoma, epidural hematoma, subarachnoid hematoma, increase in intracranial pressure, ischemia and shock. In certain embodiments, administration of NACA leads to the alleviation and/or improvement of one or more of these symptoms.

In certain embodiments, administration of NACA is useful for treating secondary pathologies associated with the primary penetrating head injury. These injuries can include a secondary traumatic brain injury of any kind. The secondary traumatic brain injury can include concussion, contusion or laceration. The secondary traumatic brain injury can be of any severity. The Secondary traumatic brain injury can be mild, moderate or severe. The Secondary traumatic brain injury can be accompanied by cerebral hemorrhage. Cerebral hemorrhage includes epidural hematoma, subdural hematoma, subarachnoid hemorrhage, and intraventricular hemorrhage. Symptoms of the secondary traumatic brain injury include headache, vomiting, nausea, lack of motor coordination, dizziness, difficulty balancing, lightheadedness, blurred vision or tired eyes, ringing in the ears, bad taste in the mouth, fatigue or lethargy, changes in sleep patterns, convulsions, an inability to awaken, dilation of one or both pupils, slurred speech, aphasia, dysarthria, weakness or numbness in the limbs, loss of coordination, confusion, restlessness, agitation, changes in appropriate social behavior, deficits in social judgment, cognitive changes, problems with sustained attention, processing speed, and executive functioning and alexithymia. NACA can be used to treat any of these types of traumatic brain injuries and to improve any of the symptoms associated with traumatic brain injury.

According to certain embodiments, NACA or derivatives thereof can be administered systemically or on, in or near the site of the wound caused by the penetrating head injury. Systemic administration methods include intraperitoneal, intravenous, oral or transdermal administration. Administration onto the site of the wound can include injection or transdermal delivery.

Doses, amounts or quantities of NACA, or derivatives thereof, are determined on an individual basis. As is appreciated by the skilled practitioner in the art, dosing is dependent on the severity and responsiveness of the penetrating head injury to be treated, but will normally be one or more doses per day, with course of treatment lasting from several days to several months, or until a cure is effected or a diminution of disease state is achieved. Persons ordinarily skilled in the art can easily determine optimum dosages, dosing methodologies and repetition rates. For example, a pharmaceutical formulation for orally administrable dosage form can comprise NACA, or a pharmaceutically acceptable salt, ester, or derivative thereof in an amount equivalent to at least 25-500 mg per dose, or in an amount equivalent to at least 50-350 mg per dose, or in an amount equivalent to at least 50-150 mg per dose, or in an amount equivalent to at least 25-250 mg per dose, or in an amount equivalent to at least 50 mg per dose. NAC amide or a derivative thereof can be administered to both human and non-human mammals. It therefore has application in both human and veterinary medicine. In certain embodiments, treatment with NACA can last for 1, 2, 3, 4, 5, 10, 15, 20, 25, 30, 35, 40, 45 or 50 weeks.

Pharmaceutical Compositions

As used herein the term “pharmaceutical composition” refers to a preparation of one or more of the components described herein, or physiologically acceptable salts or prodrugs thereof, with other chemical components such as physiologically suitable carriers and excipients. The purpose of a pharmaceutical composition is to facilitate administration of a compound to an organism. The term “prodrug” refers a precursor compound that can hydrolyze, oxidize, or otherwise react under biological conditions (in vitro or in vivo) to provide the active compound. Examples of prodrugs include, but are not limited to, metabolites of NSAIDs that include biohydrolyzable moieties such as biohydrolyzable ainides, biohydrolyzable esters, biohydrolyzable carbamates, biohydrolyzable carbonates, biohydrolyzable ureides, and biohydrolyzable phosphate analogues.

The term “excipient” refers to an inert or inactive substance added to a pharmaceutical composition to further facilitate administration of a compound. Non-limiting examples of excipients include calcium carbonate, calcium phosphate, various sugars and types of starch, cellulose derivatives, gelatin, vegetable oils and polyethylene glycols.

The pharmaceutical compositions of the present invention comprise NAC Amide or derviate thereof and may also include one or more additive drugs (e.g., additional active ingredients), such as, but not limited to, NSAIDs, antibiotics, conventional anti-cancer and/or anti-inflammatory agents that may be suitable for combination therapy.

The pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or by lyophilizing processes.

The compositions for use in accordance with the present invention thus may be formulated in conventional manner using one or more pharmaceutically acceptable carriers comprising excipients and auxiliaries, which facilitate processing of the active compounds into preparations which can be used pharmaceutically. Proper formulation is dependent upon the route of administration chosen.

The term “administration” or any lingual variation thereof as used herein is meant any way of administration. The one or more of NAC Amide or derivative thereof and at least one additional drug may be administered in one therapeutic dosage form or in two separate therapeutic dosages such as in separate capsules, tablets or injections. In the case of the two separate therapeutic dosages, the administration may be such that the periods between the administrations vary or are determined by the practitioner. It is however preferred that the second drug is administered within the therapeutic response time of the first drug. The one or more of NAC Amide or derivative thereof and at least one additional drug which may be administered either at the same time, or separately, or sequentially, according to the invention, do not represent a mere aggregate of known agents, but a new combination with the valuable property that the effectiveness of the treatment is achieved at a much lower dosage of said at least one additional drug.

The pharmaceutical compositions of the present invention may be administered by any convenient route, for example, by infusion or bolus injection, by absorption through epithelial or mucocutaneous linings (e.g., oral mucosa, rectal and intestinal mucosa, etc.) and may be administered together with any other therapeutic agent. Administration can be systemic or local.

Various delivery systems are known, e.g., encapsulation in liposomes, microparticles, microcapsules or capsules, that may be used to administer the compositions of the invention. Methods of administration include but are not limited to intradermal, intramuscular, intraperitoneal, intravenous, subcutaneous, epidural, oral, sublingual, intranasal, intracerebral, intravaginal, transdermal, rectally (including by suppository or enema), by inhalation, or topically to the cars, nose, eyes, or skin. The preferred mode of administration is left to the discretion of the practitioner, and will depend in part upon the site of the medical condition (such as the site of cancer) and the severity of thereof.

For example, for injection the composition of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example DMSO, or polyethylene glycol are generally known in the art.

For oral administration, the composition can be formulated readily by combining the active components with any pharmaceutically acceptable carriers known in the art. Such “carriers” may facilitate the manufacture of such as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum, methyl cellulose, hydroxypropylmethyl-cellulose, sodium carbomethylcellulose, and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active components may be dissolved or suspended in suitable liquids, such as fatty oils, liquid paraffin, or liquid polyethylene glycols.

Dyestuffs or pigments may be added to the tablets or dragee coatings for identification or to characterize different combinations of active NSAID doses. In addition, stabilizers may be added.

Pharmaceutical compositions for parenteral administration include aqueous solutions of the active preparation in a water-soluble form. Additionally, suspensions of the active preparation may be prepared as oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl, cellulose, sorbitol or dextran. Optionally, the suspension may also contain suitable stabilizers or agents, which increase the solubility of the compounds, to allow for the preparation of highly concentrated solutions.

Alternatively, the composition may be in a powder form for constitution before use with a suitable vehicle, e.g., sterile, pyrogen-free water. The exact formulation, route of administration and dosage may be chosen by the physician familiar with the patient's condition. (See for example Fingl, et al., 1975, in “The Pharmacological Basis of Therapeutics”, Chapter I, p. 1). Depending on the severity and responsiveness of the condition treated, dosing can also be a single administration of a slow release composition, with course of treatment lasting from several days to several weeks or until cure is effected or diminution of the disease state is achieved.

### EXAMPLES

### Example 1: NACA Reduces Neural Degeneration in Rats with Penetrating Head Injury

A penetrating brain injury (non-fatal) was produced in rats. A needle was used to create a penetrating brain injury in rats. The rats were studied 24 hours post trauma. 5 rats received NACA (300 mg ip per kg body weight and a bolus after 4 hours) and were compared to 5 controls (i.e. trauma, no NACA). Significant increased survival of cortical neurons was shown in the rats administered NACA than controls. This was verified by Fluoro Jade staining of histological samples. Fluoro Jade stains degenerating neurons, giving the number of dying neurons. Among the NACA-treated group the number of degenerating cells ranged between 200 and 450, as compared to the untreated group of between 450 and 800.

### Example 2: Characterization of Neural Tissue in Rats with Penetrating Head Injury Administered NACA

Penetrating head injury is the next result of a primary injury, in this case the needle penetration and a secondary injury, which involves cytokines and inflammation. The presence and relative amounts of certain inflammatory cytokines and other signaling molecules involved with inflammation will be measured to investigate the mechanism of the protective effect of NACA. These include p38 mitogen-activated protein kinase (MAPK) and inducible nitric oxide synthase (iNOS) signaling, as well as Bcl-2, Bax, and NF-κB expression.

### Example 3. Immunohistochemical Analysis of Brain Tissue of Rats with Penetrating Head Injury when Administered NACA

Focal traumatic brain injuries (TBIs) using a penetrating brain injury model were produced in male rats (n=5). After 2 h and 24 h the brains were removed, cut in 14 nm coronal sections and subjected to immunohistochemical analyses.

Results

The level of fluoro jade stained neuronal degeneration was lower in the group treated with NACA after 24 h. iNOS was unregulated in the perilesional area, not differing between groups. Oxidative stress detected by peroxynitrite surrogate marker 3-NT was detected in the perilesional area, not differing between groups.

What is claimed is:

1. A method of treating penetrating head injury in a subject in need thereof comprising administering to the subject a composition comprising a therapeutically effective amount of N-acetylcysteine amide (NACA).

2. The method of claim 1, wherein the composition further comprises a pharmaceutically acceptable salt or excipient.

3. The method of claim 1, wherein the treatment of the penetrating head injury comprises penetration of the skin, the outer layer of the skull, the inner layer of the skull, the dura mater or the brain.

4. The method of claim 1, wherein the penetration is caused by a gunshot.

5. The method of claim 1, wherein the NACA is administered systemically.

6. The method of claim 5, wherein the NACA is administered intraperitoneally, intravenously, orally or transdermally.

7. The method of claim 1, wherein the NACA is administered directly onto or into a wound caused by the penetrating head injury.

8. The method of claim 1, wherein the subject is a mammal.

9. The method of claim 8, wherein the mammal is a human.

10. The method of claim 1, wherein the NACA is administered at between 5 and 10,000 mg/kg.

11. The method of claim 1, wherein the NACA is administered at between 75 and 600 mg/kg to the subject.

12. The method of claim 11, wherein the NACA is administered at between 200 and 400 mg/kg to the subject.

13. The method of claim 3, wherein the brain tissue comprises synaptic cells.

14. The method of claim 13, wherein RCR and/or respiration rate is increased in the synaptic cells.

15. The method of claim 3, wherein the brain tissue tissue comprises non-synaptic cells.

16. The method of claim 15, wherein RCR and/or respiration rate is increased in the non-synaptic cells.

17. The method of claim 15, wherein the non-synaptic cells comprise neuronal soma cells.

18. The method of claim 15, wherein the non-synaptic cells comprise glial cells.

19. The method of claim 1, wherein a secondary traumatic brain injury is also treated.

20. The method of claim 19, wherein the secondary traumatic brain injury is concussion.
